(19) United States
(12) Patent Application Publication
(10) Pub. No.: US 2023/0233104 A1
(43) Pub. Date: Jul. 27, 2023

(54) METHODS AND SYSTEMS FOR CAPTURING AND VISUALIZING SPINAL MOTION

(71) Applicant: Arizona Board of Regents on Behalf of Arizona State University, Scottsdale, AZ (US)

(72) Inventors: Jorge Caviedes, Mesa, AZ (US); Baoxin Li, Chandler, AZ (US); Pamela Swan, Chandler, AZ (US); Jiuxu Chen, Tempe, AZ (US)

(21) Appl. No.: 18/191,268

(22) Filed: Mar. 28, 2023

Related U.S. Application Data
(63) Continuation of application No. PCT/US2021/063301, filed on Dec. 14, 2021.
(60) Provisional application No. 63/188,736, filed on May 14, 2021.
(60) Provisional application No. 63/125,772, filed on Dec. 15, 2020.

(51) Int. Cl. A61B 5/11; A61B 5/00

(52) U.S. Cl. CPC A61B 5/1116; A61B 5/1121; A61B 5/7271; A61B 2562/04

(57) ABSTRACT

Exemplary embodiments of wearable stretch sensors and applications of using the same are disclosed. In embodiments, the sensors and the applications disclosed herein can be used to capture spinal motion and posture information.

### CROSS-REFERENCE TO RELATED APPLICATIONS

This application is a continuation of PCT Patent Application No. PCT/US2021/063301 filed on Dec. 14, 2021, now WIPO Patent Application Publication WO/2022/132764 entitled “Methods and Systems for Capturing and Visualizing Spinal Motion.” PCT/US2021/063301 claims priority to U.S. Provisional Application No. 63/188,736 filed on May 14, 2021 and entitled “Methods and Systems for Capturing and Visualizing Spinal Motion,” and to U.S. Provisional Application No. 63/125,772 filed on Dec. 15, 2020 and entitled “Methods and Systems for Capturing and Visualizing Spinal Motion.” The disclosures of each of the foregoing applications are hereby incorporated by reference in their entirety, including but not limited to those portions that specifically appear hereinafter, but except for any subject matter disclaimers or disavowals, and except to the extent that the incorporated material is inconsistent with the express disclosure herein, in which case the language in this disclosure shall control.

### TECHNICAL FIELD

The present disclosure relates to sensors, and in particular to wearable stretch sensors for capturing and visualizing spinal motion.

### BACKGROUND

Motion biofeedback uses motion sensors, for example for posture, position feedback, or pain management. The motion sensors can generate position-driven alarms to the user. Augmented reality and physio games can be applied in the context of motion biofeedback to help manage pain and aid in exercise motivation. Unlike position feedback, biomechanical feedback requires linking sensors with personalized biomechanical models. Thus, biomechanical feedback links dynamic surface landmarks' monitoring to highly accurate biomechanics.

Currently, there is no technology capable of capturing spinal motion and posture through use of a wearable stretch sensor array. To meet this need, disclosed herein are various aspects and embodiments of wearable stretch sensor arrays that can be used in motion and posture monitoring.

### SUMMARY

In an exemplary embodiment, a system to monitor and visualize spine motion is provided, comprising: a scanning device capable of capturing subject/patient specific parameters of spinal curvature, range of motion of the spine, and regional and segmental angles of the spine; at least one sensor capable of processing signals resulting from spinal movement; and a device capable of producing a 3D model of the spine based on the parameters captured by the scanning device and the signals processed by the at least one sensor.

In embodiments, the scanning device is invasive. In embodiments, the scanning device is non-invasive.

In embodiments, the 3D model of the spine comprises a 3D angular position of the spine. In embodiments, the system further comprises an analytics device capable of interpreting the signals processed by the at least one sensor. In embodiments, the analytics device produces the 3D angular position of the spine based on interpreting the signals processed by the at least one sensor. In embodiments, the 3D model of the spine is capable of producing visual biofeedback to a user of the system. In embodiments, the system further comprises a monitoring device that produces data based on the interpretation by the analytics device of the signals produced by the at least one sensor. In embodiments, the data that is produced results in visual biofeedback to a user of the system. In embodiments, the at least one sensor comprises a sensor array. In embodiments, the at least one sensor comprises at least four (4) capacitive stretch sensors. In embodiments, the at least four (4) capacitive stretch sensors are in an X-shaped configuration. In embodiments, the system further comprises a display configured to produce visual feedback of the spinal movement and the 3D model. In embodiments, the display is part of a VR headset. In embodiments, the regional and segmental angles of the spine derive from the thoracolumbar axis, i.e., the line between vertebrae C7 to S1.

In another exemplary embodiment, a device attached to the back of a subject to measure spine motion and spine curvature change is provided, the device comprising a first capacitive stretch sensor located on the top left of the back of the subject; a second capacitive stretch sensor located on the top right of the back of the subject; a third capacitive stretch sensor located on the lower right of the back of the subject; and a fourth capacitive stretch sensor located on the lower left of the back of the subject.

In embodiments, the first capacitive stretch sensor, the second capacitive stretch sensor, the third capacitive stretch sensor and the fourth capacitive stretch sensor are built into a wearable garment or clothing. In embodiments, the first capacitive stretch sensor, the second capacitive stretch sensor, the third capacitive stretch sensor and the fourth capacitive stretch sensor are in a X-shaped configuration. In embodiments, the first capacitive stretch sensor is attached to a left shoulder area of a wearer, the second capacitive stretch sensor is attached to a right shoulder area of the wearer, the third capacitive stretch sensor is attached to a right anterior superior iliac spine area of the wearer, and the fourth capacitive stretch sensor is attached to a left anterior superior iliac spine area of the wearer.

In another exemplary embodiment, a method of computing spine angle positions from a neutral standing posture is provided comprising: measuring spinal axial reference angles in 3D (Ax, Ay, and Az); and measuring geometric distortions of the quadrilateral surface spanned by the X-shaped sensor array in 3D (Dx, Dy, and Dz).

In embodiments, the method further comprises determining a proportionality constant that links the spinal axial reference angles and the geometric distortions of the spinal axial reference angles. In embodiments, determining the proportionality constant comprises estimating spinal reference angles of Ax, Ay, and Az, and estimating corresponding distortions of Dx, Dy, and Dz.

In embodiments, the axial angles for each 3D axis are computed by the following equations: X_Axis=0.5*[1+(Dx/Ax_max)*(Ax_r/Dx_r)]; Y_Axis=0.5*[1+(Dy/Ay_max)*(Ay_r/Dy_r)]; and Z_Axis=0.5*[1+(Dz/Az_max)*(Az_r/Dz_r)]. In embodiments, the maximum angles are in the range of −60° to 60°. In embodiments, the equations normalize angular values to a range between 0.0 and 1.0. In embodiments, a neutral posture is around 0.05.

In another exemplary embodiment, a method to compute the transfer functions between sensor data, geometric distortions of the sensor array, and angular positions of the spine is provided, the method comprising the solutions for [A], as defined herein, [M], as defined herein, and/or a direct estimation of [A]×[M], as defined herein.

In another exemplary embodiment, a method to provide biofeedback is provided, the method comprising monitoring deviations from recorded angular positions from a correct exercise execution in real time and at the end of an exercise.

In another exemplary embodiment, a method to incorporate visual cues and attentional cues to the 3D visualization is provided comprising conveying corrective data and reinforcing a type of biofeedback by adding correctness information to the visualization in real time.

In another exemplary embodiment, a linear model of the dependency between four stretch sensor signals and the distortions is provided, comprising one or more of the following equations: Dx=(ΣSi)/2, i=1-4; Dy=(S1+S3)−(S2+S4); Dz=(S1+S4)−(S2+S3); X_Axis=0.5*[1+(Dx/Ax_max)*(Ax_r/Dx_r)]; Y_Axis=0.5*[1+(Dy/Ay_max)*(Ay_r/Dy_r)]; and Z_Axis=0.5*[1+(Dz/Az_max)*(Az_r/Dz_r)].

In another exemplary embodiment, a model based on the 2D projective transformation parameters of the sensor array quadrilateral and the angular positions of the spine is provided, comprising one or more of the follow equations:

$$H = [ \begin{matrix}{a}_{11} & {a}_{12} & {t}_{z} \\ {a}_{21} & {a}_{22} & {t}_{y} \\ {v}_{1} & {v}_{2} & v\end{matrix} ] = ( \begin{matrix}A & t \\ {v}^{T} & v\end{matrix} ) ; H = [ \begin{matrix}sR & t \\ {0}^{T} & 1\end{matrix} ] [ \begin{matrix}k & 0 \\ {0}^{T} & 1\end{matrix} ] [ \begin{matrix}λ & 0 \\ {0}^{T} & 1\end{matrix} ] [ \begin{matrix}1 & 0 \\ {V}^{T} & v\end{matrix} ] ; and [ \begin{matrix}{x}^{′} \\ {y}^{′} \\ 1\end{matrix} ] = [ \begin{matrix}{a}_{11} & {a}_{12} & {t}_{z} \\ {a}_{21} & {a}_{22} & {t}_{y} \\ {v}_{1} & {v}_{2} & v\end{matrix} ] \times [ \begin{matrix}x \\ y \\ 1\end{matrix} ] .$$

In embodiments, (x,y) are the 2D coordinates of quadrilateral vertices in neutral posture, and (x′,y′) are the 2D coordinates of the same vertices under motion-induced geometric distortion conditions.

### BRIEF DESCRIPTION OF THE DRAWINGS

With reference to the following description and accompanying drawings:

FIG. 1A illustrates an exemplary device of four wearable stretch sensors attached to the back of a subject in accordance with various exemplary embodiments.

FIG. 1B illustrates the spinal axis, and FIG. 1C illustrates an exemplary embodiment of monitoring various points of the sensor array area during movement.

FIG. 2 illustrates an exemplary graph of sensor signal changes for a mono-axis motion sequence in accordance with various exemplary embodiments.

FIG. 3 illustrates an exemplary system for monitoring and visualizing spinal motion in accordance with various exemplary embodiments.

FIG. 4 illustrates an exemplary 3D spine model animation view of the subject, and a graph of sensor signals and angular positions for validation of exercise by a physical therapist or expert in accordance with various exemplary embodiments.

FIG. 5 illustrates an exemplary graph of spine angular position changes in degrees and sensor signal changes during a sequence of four biaxial motions in accordance with various exemplary embodiments.

FIG. 6 illustrates an exemplary framework including 3D spine model animation, spine angular positions and their corresponding video recordings for system validation using a sequence of biaxial motions in accordance with various exemplary embodiments.

FIG. 7 illustrates projective transformations of the sensor signals between neutral posture and spinal motions in accordance with various exemplary embodiments.

FIG. 8 illustrates an exemplary embodiment of an inertial measurement unit (IMU) sensor.

FIG. 9 illustrates stretch sensors to monitor motion of the lumbar spine.

FIG. 10 illustrates a further exemplary embodiment of an IMU sensor.

FIG. 11 illustrates an exemplary embodiment of a two-loop sensor system for posture monitoring based on position feedback.

FIGS. 12A and 12B illustrate an exemplary embodiment of linking dynamic surface landmarks monitoring to biomechanics.

FIG. 13 shows an exemplary embodiment of personalizing the spine model using parameters obtained by non-invasive optical scan.

### DETAILED DESCRIPTION

The following description is of various exemplary embodiments only, and is not intended to limit the scope, applicability or configuration of the present disclosure in any way. Rather, the following description is intended to provide a convenient illustration for implementing various embodiments including the best mode. As will become apparent, various changes may be made in the function and arrangement of the elements described in these embodiments without departing from principles of the present disclosure.

For the sake of brevity, conventional techniques and components for sensors, such as wearable stretch sensor systems, may not be described in detail herein. Furthermore, the connecting lines shown in various figures contained herein are intended to represent exemplary functional relationships and/or physical couplings between various elements. It should be noted that many alternative or additional functional relationships or physical connections may be present in exemplary wearable stretch sensor systems and/or components thereof.

Wearable stretch sensors are presently used to monitor human activity and human health. Wearable sensors of different types have been proposed as a solution to allow mobility, user friendliness, and ease of use and monitoring. It is possible to monitor physiological parameters as well as biomechanical activity using pressure and strain sensors. Accurate real time 3D motion capture of the human spine is of interest for medical diagnosis and rehabilitation of postural disabilities. A motion sensing system comprised of 3 inertial measurement units (IMUs) attached to the head, torso and hips has been proposed before with limited applications.

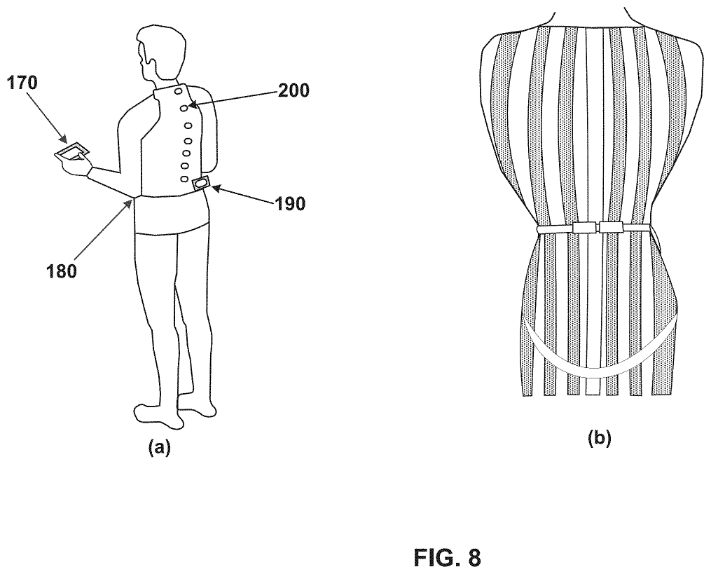

While wearable IMUs can monitor motion, electromyography (EMG) sensors have been developed to monitor physiological muscle activity. EMG biomechanical biofeedback is widely used in rehabilitation and therapeutic treatment, including cardiovascular accident rehabilitation and LBP treatment. EMG biofeedback is primarily used in sports performance improvement as part of sports psychology programs. Garments incorporating EMG sensors to monitor major muscle groups activity and performance have been benchmarked against research grade EMG systems. Stretch sensors in a triangular array have been shown to be suitable to monitor exercise correctness for scoliosis therapy and lower back exercise have been proposed by the inventors. See J. E. Caviedes, B. Li and V. C. Jammula, “Wearable Sensor Array Design for Spine Posture Monitoring During Exercise Incorporating Biofeedback,” in IEEE Transactions on Biomedical Engineering, vol. 67, no. 10, pp. 2828-2838, October 2020, doi: 10.1109/TBME.2020.2971907. As shown in FIG. 9, an array with 4 sensors has been proposed to measure spinal angles on the lumbar spine, however such system deals only with the lumbar spine and measures spine angles in single axis motion. See A. Yamamoto, et al. “Method for measuring tri-axial lumbar motion angles using wearable sheet stretch sensors,” PLOS ONE 12(10): e0183651, 2017. https://doi.org/10.1371/journal.pone.0183651.

Unsupervised spine exercise is practiced abundantly in many contexts, including fitness and therapy. There are no effective methods to monitor and supervise the spinal exercise without complex lab equipment and instruments available usually to professional sports and in-clinic therapy facilities. Mobile systems based on wearable sensors and immersive visualization are the ideal solution but only if the design meets the requirements of low complexity and usability. Accordingly, improved systems and methods are desirable.

The present disclosure is directed towards methods and devices used to capture spinal motion and posture information by means of a wearable stretch sensor array. The devices disclosed herein enable monitoring motion as well as visualizing posture of the spine. The technology disclosed herein has potential to be a core component of at home exercise and therapy programs designed by professional trainers and therapists. Biofeedback systems and methods based on the devices and methods disclosed herein have market potential. Using the technology disclosed herein, motion monitoring may be realized by analyzing the sensor signals and posture visualization is realized through a method of animating a 3D spine model in real time. In various embodiments, an exemplary device disclosed herein uses four (4) capacitive stretch sensors with a linear dependency on stretch and calibrated in elongation in millimeters. In various embodiments, the sensors disclosed herein use angles from 3-axes that are computed one at a time from the sensor signals. According to various exemplary embodiments, an in vivo system is disclosed herein using human subjects wearing spine sensing device.

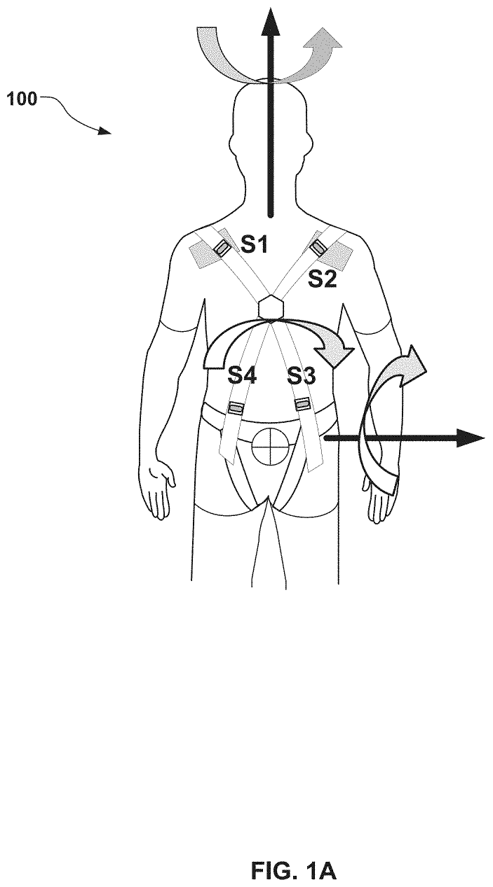

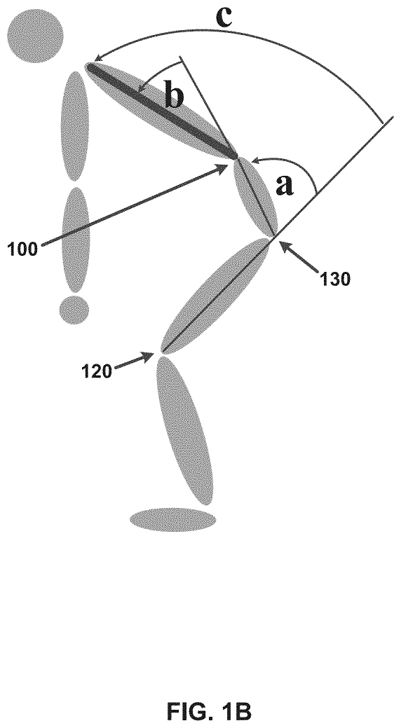

The present disclosure relates to the use of stretch sensors for motion and posture monitoring. In various embodiments, an array of four (4) sensors in an X-shaped configuration is disclosed, as illustrated in FIG. 1A. The array of four (4) sensors can be used to monitor and visualize spinal motion with accuracy and simplicity.

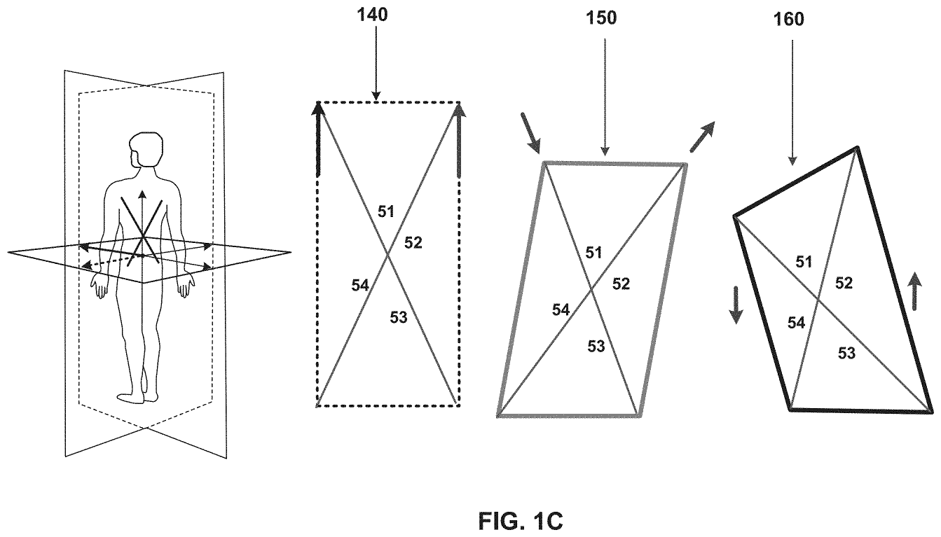

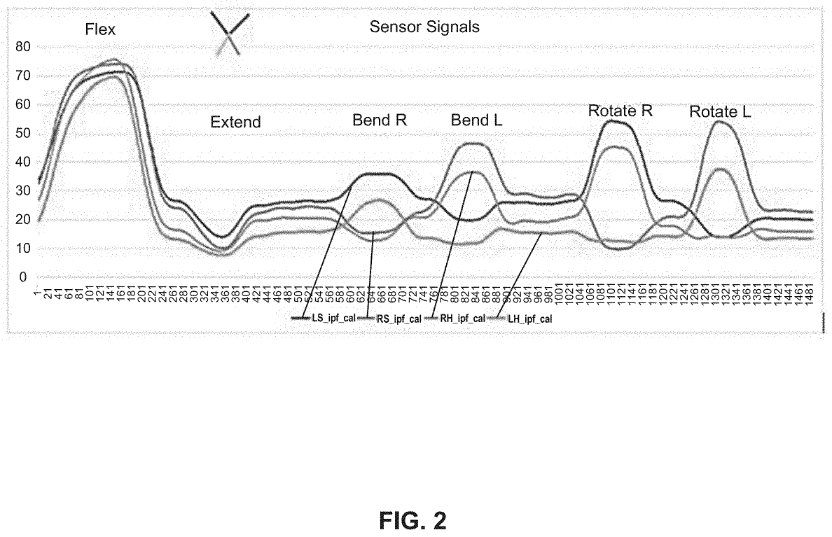

In one aspect of the present disclosure, a system comprising capacitive stretch sensors is disclosed. In various embodiments, the capacitive stretch sensors have a linear dependency on stretch. In various embodiments, the capacitive stretch sensors are calibrated in elongation in millimeters. In various embodiments, the system (100) comprises four (4) sensors which are attached to the back of a subject as illustrated in FIG. 1A. In various embodiments, the four (4) sensors detect signals caused by motion and curvature changes of the subject's spine. In various embodiments, the sensors are built into a garment or exercise clothing as illustrated in FIG. 1A. In various embodiments, the four (4) sensors are in an X-shaped configuration and attached to the left and right shoulders, and the left and right posterior superior iliac spine (PSIS), as illustrated in FIG. 1A. In various embodiments, the subject's spine angles on three axes may be determined, for example one at a time, based on computation from the sensor signals as illustrated in FIG. 1A. The three axes consist of X axis, as indicated by green lines and arrows in FIG. 1A, Y axis as indicated by red lines and arrows in FIG. 1A, and Z axis as indicated by blue lines and arrows in FIG. 1A. In various embodiments, movement of X axis captures flexion and extension on the sagittal plane, movement of Y axis captures rotation along the cranio-caudal direction, and movement of Z axis captures lateral bending on the coronal plane. In various embodiments, the signal changes of the four sensors are measured in a mono-axis spine motion sequence comprising flexion, extension, bending right, bending left, right rotation and left rotation, as illustrated in FIG. 2. In various embodiments, a simplified model is disclosed herein. The simplified model comprises a cylinder representing a subject's back with the four (4) sensors on the surface of the cylinder. In various embodiments, the simplified model further comprises a 2D representation of the cylinder's sensing surface and deformations caused by motion.

In another aspect of the present disclosure, a method of measuring an angle of spine movement is disclosed herein. In various embodiments, a measurement of an angle of lumbar spine flexion/extension is proportional to the sum of four (4) sensor signal values in which all four (4) sensors stretch uniformly in a typical case. In various embodiments, the four (4) sensors are labeled as follows: S1 for the top left sensor, S2 for the top right sensor, S3 for the lower right sensor, and S4 for the lower left sensor, as illustrated in FIG. 1A. In various embodiments, thoracolumbar rotation or Y angle causes the diagonal S1/S3 sensors to contract and the diagonal S2/S4 sensors to stretch. In various embodiments, the angle of rotation on the Y axis is a linear function of the difference in elongations of the four (4) sensors calculated by the formula: (Δ(S1+S3)−Δ(S2+S4)). In various embodiments, for the lumbar side bending or Z angle, the difference between the signals of two pairs of sensors on the left side and right side, S1/S4 and S2/S3, is linearly dependent on the angle. Thus, the Z angle is a linear function of the difference between the sum of signals of the two pairs calculated by the formula: (Δ(S1+S4)−Δ(S2+S3)).

In another aspect of present disclosure, the changes in angular positions described herein for single axis motion may be generalized to account for bi- and tri-axis motions to account for normal coupling of spinal motion (e.g. bending is coupled with some rotation) as well as complex exercises involving motion along more than one axis. The generalized relationships between the arrays of sensor signals [S], geometric distortions of the sensor array [D], and the angular positions of the main spinal axis may be the following:

$$[ \begin{matrix}{D}_{1} \\ {D}_{2} \\ {D}_{3}\end{matrix} ] = [ \begin{matrix}{A}_{11} & {A}_{12} & {A}_{13} & {A}_{14} \\ {A}_{21} & {A}_{22} & {A}_{23} & {A}_{24} \\ {A}_{31} & {A}_{32} & {A}_{33} & {A}_{34}\end{matrix} ] \times [ \begin{matrix}{S}_{1} \\ {S}_{2} \\ {S}_{3} \\ {S}_{4}\end{matrix} ] \\ [ \begin{matrix}{P}_{1} \\ {P}_{2} \\ {P}_{3}\end{matrix} ] = [ \begin{matrix}{M}_{11} & {M}_{12} & {M}_{13} \\ {M}_{21} & {M}_{22} & {M}_{23} \\ {M}_{31} & {M}_{32} & {M}_{33}\end{matrix} ] \times [ \begin{matrix}{D}_{1} \\ {D}_{2} \\ {D}_{3}\end{matrix} ]$$

where [A] is the transfer function between sensor signals and geometric distortions of the sensor array, and [M] is the transfer function between geometric distortions and angular positions [P]. In the case of single axis motion [A] is:

$$[ \begin{matrix}{A}_{11} & {A}_{12} & {A}_{13} & {A}_{14} \\ {A}_{21} & {A}_{22} & {A}_{23} & {A}_{24} \\ {A}_{31} & {A}_{32} & {A}_{33} & {A}_{34}\end{matrix} ] = [ \begin{matrix}C & C & C & C \\ 1 & - 1 & - 1 & 1 \\ 1 & - 1 & 1 & - 1\end{matrix} ]$$

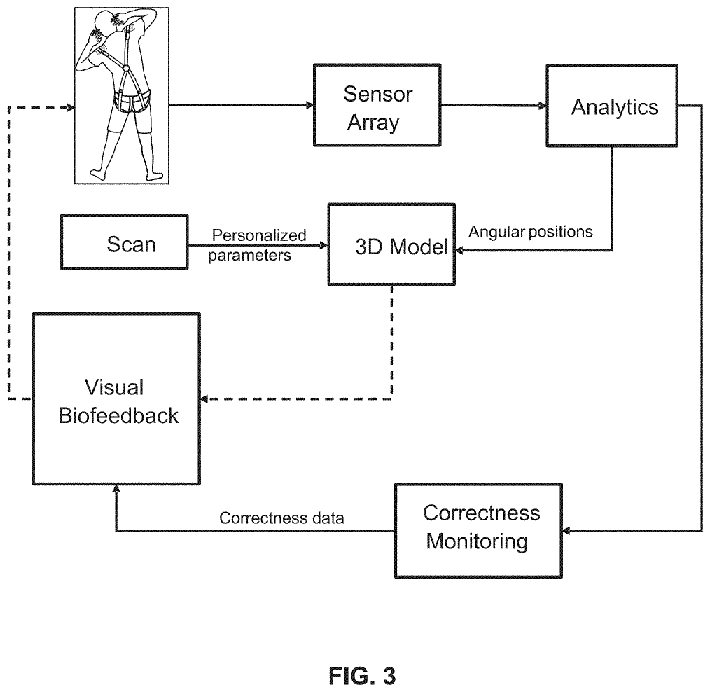

C=½ (corresponding to the equations presented before)
The angular positions [P] may be sent to the 3D spine model for dynamic visualization as shown in FIG. 3. For the general case of multi-axis motion, the solutions to [A] and [M] may be obtained by regression methods using experimental data and ground truth angle measurements. Moreover, a direct solution may also be obtained for [M]×[A] using the same method described herein.

In various embodiments, the angle calibration with respect to the ground truth is carried out by measuring the subject's range of motion on each axis and finding the linear dependency between angle and sensor readings for each case. In various embodiments, angles are measured by a variety of techniques including computer vision, image analytics, and spine goniometers. In various embodiments, angles are measured by a method using analytics on photos taken while the subject's spinal segments (e.g. C7, L4, S2) are visualized using optical markers.

In various embodiments, the four-sensor array has additional advantages for personalization. The initial posture of individuals may not be perfectly symmetric, while the ratio values of S1/S4 and S2/S3 can be used to determine symmetry. When S1/S4 is equal to S2/S3, there is symmetry. In various embodiments, the ratios of S1/S4 and S2/S3 are used as an indicator of correctness and also as a parameter for the spine model animation when the exercises require symmetry. This is a unique advantage to personalize the system for subjects with conditions such as scoliosis, lordosis, kyphosis, and so forth. For normal subjects, the spine may have a double curve sometimes modeled as a cubic spline.

In another aspect of the present disclosure, a system to monitor and visualize spine motion is disclosed. In embodiments, the system includes monitoring and visualization components supported by analytics and a personalized spine model, as illustrated in FIG. 3. In various embodiments, the spine model is personalized by means of a noninvasive scan to capture parameters such as spinal curvature, range of motion, and regional and segmental angles of the spine from vertebrae C7 to S1 and import them into the spine model. The spine model may be based on a finite element approach such that the angular position in 3D as well as the actual spine deformation corresponds to the geometric deformations of the sensor array and captured in the transfer functions [A] and [M] for the general case of multi-axis motion.

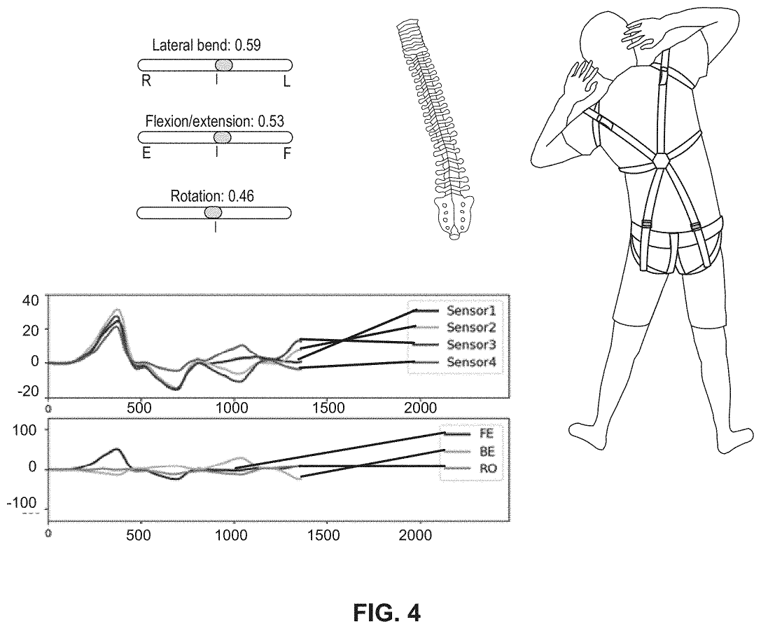

In another aspect of the present disclosure, a method for validation of exercise by physical therapist or expert is disclosed herein. In embodiments, the method utilizes a 3D spinal model animation and graph of sensor signals, in a direct or captured view of the subject, and angular positions for real time visualization of the sensor, geometric distortion, and/or angular positions which are interpreted by physical therapists and trainers to allow continuous system updates, as illustrated in FIG. 4.

In another aspect of the present disclosure, a method of computing spine geometric distortions from their neutral posture based on sensor signal changes is disclosed herein. In embodiments, the spine distortions, Dx, Dy and Dz, induced by flexion-extension, rotation, and bending, respectively, may be computed based on the signal readings, Si with i∈{1,4}, of the four sensors based on Equations (1), (2) and (3). In embodiments, the spine geometric distortions (Dx, Dy, Dz) may also be identified as DFE, DRO, and DBE for distortions caused by rotations around the three spine axes during flexion/extension (FE), rotation (RO) and bending (BE) motions, respectively.

Dx=(ΣSi)/2,i=1-4 Equation (1)

Dy=(S1+S3)−(S2+S4) Equation (2)

Dz=(S1+S4)−(S2+S3) Equation (3)

In another aspect of the present disclosure, a method of computing spine angle positions from their neutral posture based on spine geometric distortions is disclosed herein. In embodiments, the spinal axial angles in 3D, Ax, Ay and Az, may be proportional to their geometric distortions, Dx, Dy and Dz. In embodiments, the proportionality constants (Ai/Di) that link angles and geometric distortions are computed by estimating three ground truth reference angles Ax_r, Ay_r and Az_r and the corresponding distortions Dx_r, Dy_r and Dz_r. In embodiments, the three spinal axial angles may be computed based on Equations (4), (5) and (6).

X_Axis=0.5*[1+(Dx/Ax_max)*(Ax_r/Dx_r)] Equation (4)

Y_Axis=0.5*[1+(Dy/Ay_max)*(Ay_r/Dy_r)] Equation (5)

Z_Axis=0.5*[1+(Dz/Az_max)*(Az_r/Dz_r)] Equation (6)

In embodiments, the computation models in Equations (4)-(6) may allow the maximum angles to be in the range of −60° to 60°, or Ax_max of +/−60°, Ay_max of +/−60°, and Az_max of +/−60°. In embodiments, the actual values for each subject may be estimated using manual goniometry. In embodiments, Equations (4)-(6) may normalize the angular values to the range 0.0-1.0, with 0.5 for the neutral posture. In embodiments, X_Axis, Y_Axis and Z_Axis may be the normalized flexion-extension, rotation, and bending angles respectively. In embodiments, the constant Ax_r/Dx_r for extension may be about one fourth (¼) of the value for flexion. In embodiments, other cases can be considered symmetric as a first approximation, but for increased precision they may be estimated separately for positive and negative angles. In embodiments, four pairs of reference angle-geometric distortion values may be obtained for each subject. An example of data values for one subject is shown in Table 1. In embodiments, negative angles and distortions for bending and rotation are assumed to be symmetrical, but can be taken separately. In embodiments, the maximum distortions allowed by the model may have been calculated by linear interpolation.

TABLE 1
Examples of personalized reference pairs of
angle-distortion values for Equations (4)-(6).

| | Max | Axis | Angle | Distortion | Max |
|---|---|---|---|---|---|
| | Angle | Value | (A) | (D) | Distortion |
| Flex | 60 | 0.0-1.0 | 40 | 70.9575 | 106.4362552 |
| Bending | 60 | 0.0-1.0 | 30 | 22.85227 | 45.70454461 |
| Rotation | 60 | 0.0-1.0 | 30 | 29.87727 | 59.75454894 |
| Extend | −60 | 0.0-0.5 | −40 | −20.6834 | −31.02511887 |

In another aspect of the present disclosure, a validation framework for the quadrangle sensor array through biaxial and triaxial motion sequences is disclosed. In embodiments, this 3-way cross validation framework (illustrated in FIG. 4) may use the data graphs, the 3D spine model animation, and the recorded video. In embodiments, the two main outputs, namely the graphical data and the spine model animation, may be validated against their corresponding video recordings. In embodiments, the angular positions may be shown in the normalized 0-1 range (−60° to +60°, with neutral posture at 0.5) used for input to the spine model. In embodiments, playbacks of the video, spine animation and graphs may be synchronized and normalized to the same time scale.

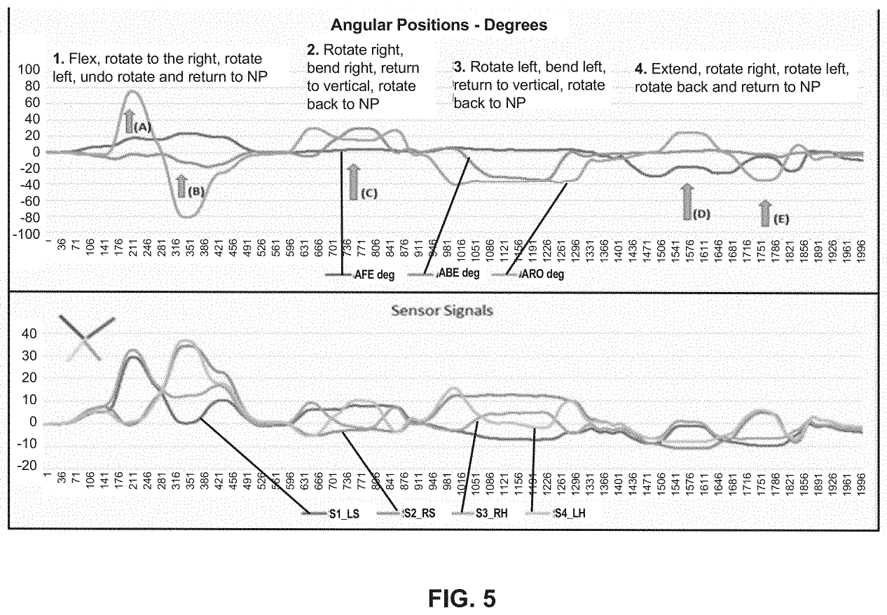

In embodiments, three sets of biaxial motions were executed in a test for validation of the quadrangle sensor design. The graphs for the test include a sequence of four (4) biaxial motions, as shown in FIG. 5. The graph of angular positions in degrees and the four (4) motion descriptions labels is shown at the top of FIG. 5 while the graph of sensor signals is shown at the bottom of the FIG. 5. Using the descriptions of the four (4) motions, one may recognize the associated sensor signal patterns and angular positions, as shown in FIG. 5. In embodiments, this description can be applied to all possible biaxial and triaxial motion cases.

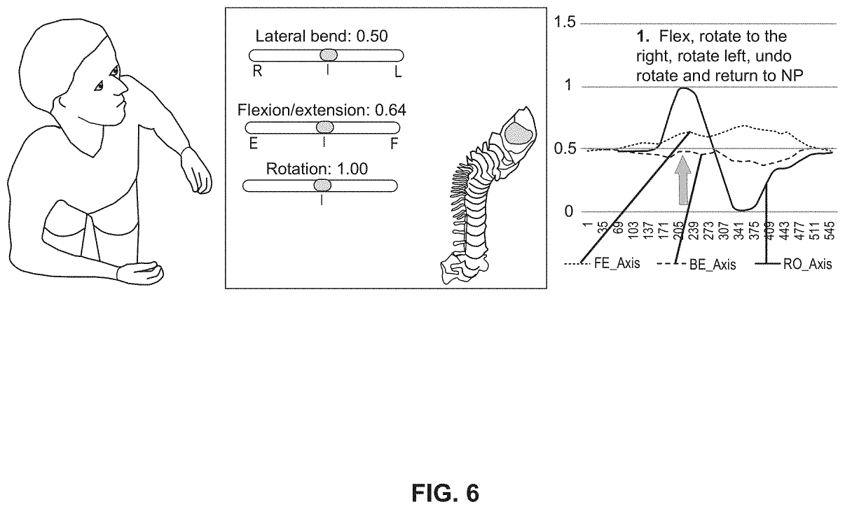

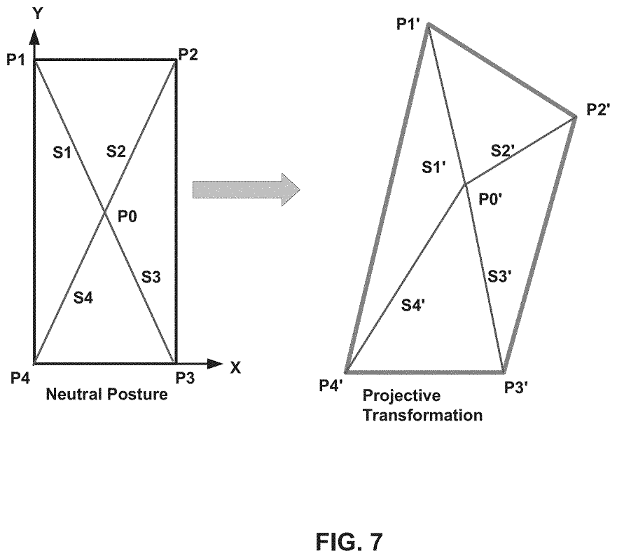

In embodiments, a validation and analysis of angular positions computed by the linear model was conducted using the motion consisting of rotation to the left while flexing by 20°, as shown in FIG. 6. The photo in the left panel of FIG. 6 was taken at a point of combined flexion plus rotation to the right that is marked by the arrow noted in the right panel. Flexion before rotation was at the hip joint instead of the lumbosacral joint and appeared to gradually increase during rotation as indicated by the line noted in the right panel. The initial attenuation of the flexion signal in this case may not be caused by the linear model, but due to the execution technique. In embodiments, as shown in FIG. 7, a projective transformation can be simulated using the sensors signals that result from spinal motions from a neutral posture.

In another aspect of the present disclosure, a model based on the 2D projective transformation parameters and the angular positions of the spine is disclosed herein. FIG. 7 shows the general formulation of the projective transformations in sensor space. S1, S2, S3 and S4 are the physical dimensions of the sensor array in neutral posture, and S1′, S2′, S3′ and S4′ are the dimensions at any point during spinal motion. Points P3 and P4 are fixed. The projective transformation [H] between neutral and modified posture can be expressed as Equation (7):

$$H = [ \begin{matrix}{a}_{11} & {a}_{12} & {t}_{z} \\ {a}_{21} & {a}_{22} & {t}_{y} \\ {v}_{1} & {v}_{2} & v\end{matrix} ] = ( \begin{matrix}A & t \\ {v}^{T} & v\end{matrix} ) \qquad Equation ( 7 )$$

A is a 2×2 non-singular matrix, t is a translation vector (zero in this case), and v=(v1, v2)^{T}. H can be decomposed as:

$$H = [ \begin{matrix}sR & t \\ {0}^{T} & 1\end{matrix} ] [ \begin{matrix}k & 0 \\ {0}^{T} & 1\end{matrix} ] [ \begin{matrix}λ & 0 \\ {0}^{T} & 1\end{matrix} ] [ \begin{matrix}1 & 0 \\ {V}^{T} & v\end{matrix} ] \qquad Equation ( 8 )$$

In this case we have three degrees of freedom. s is overall scale, k is shear, λ and 1/λ are the x and y scaling factors. H can be solved from:

$$[ \begin{matrix}{x}^{′} \\ {y}^{′} \\ 1\end{matrix} ] = [ \begin{matrix}{a}_{11} & {a}_{12} & {t}_{z} \\ {a}_{21} & {a}_{22} & {t}_{y} \\ {v}_{1} & {v}_{2} & v\end{matrix} ] \times [ \begin{matrix}x \\ y \\ 1\end{matrix} ] \qquad Equation ( 9 )$$

where (x,y) are the 2D coordinates of quadrilateral vertices in neutral posture and x′, y′) are the coordinates for any other posture. If we set the origin at P4, the three points P1, P2 and P3 can be used to solve [H].

In embodiments, a test was conducted to measure the physical dimensions of the sensor array and solve for H using a set of biaxial motions shown in Table 2. The observed relationship between motions and projective transformation parameters of Equation (8) are also shown in Table 2. The labels C, inc., dec., mean constant, increase and decrease.

TABLE 2
Relationships between spine motions and
projective transformation parameters.

| | s | λ | k |
|---|---|---|---|
| Flex/Extend | >1/<1 | <1/>1 | =0/=0 |
| Bend R/L | >1/<1 | >1/<1 | >0/<0 |
| Rotate R/L | =c/=c | =c/=c | <0/>0 |
| Flex + Bend R | inc. | inc. | inc. |
| Flex + Bend L | dec. | dec. | dec. |
| Extend + Bend R | inc. | c | inc. |
| Extend + Bend L | dec. | c | dec. |
| Flex + Rotate R | inc. | c | dec. |
| Flex + Rotate L | inc. | c | inc. |
| Extend + Rotate R | dec. | inc. | dec. |
| Extend + Rotate L | dec. | dec. | inc. |
| Bend R + Rotate R | inc. | dec. | dec. |
| Bend R + Rotate L | dec. | dec. | inc. |
| Bend L + Rotate R | inc. | c | dec. |
| Bend L + Rotate L | dec. | c | inc. |

In another aspect of the present disclosure, methods and apparatus disclosed herein are used as the core of mobile, at home exercise, and/or therapy programs designed by professional trainers and therapists. The visual biomechanical biofeedback may use any type of display, including immersive VR glasses with semi-transparent display.

In embodiments, the stretch sensors disclosed herein are suitable for integration into smart textile/clothing. In embodiments, the stretch sensors disclosed herein are less demanding in terms of signal and processing complexity relative to other wearable sensors on the market. Table 3 compares stretch sensors with inertial measurement unit (IMU) and electromyography (EMG) sensors in terms of (i) whether the different types of sensors are unobtrusive with textiles/clothing and (ii) signal and processing complexity of the sensors. As shown by the three (3) asterisks with the stretch sensors, relative to the two (2) asterisks of the IMU and EMG sensors, the stretch sensor is less conspicuous. As shown one (1) asterisk of the of the stretch sensors relative to the three (3) and two (2) asterisks of the IMU and EMG sensors, respectively, the stretch sensors are less complex in terms of signaling and processing.

TABLE 3

| | | Textile/Clothing | Signal & Processing |
|---|---|---|---|
| Type of Sensor | Principle | Unobtrusive | Complexity |
| IMU | Gyro | ** | *** |
| EMG | Elec. Sensor | ** | ** |
| Stretch | Capacitive | *** | * |

Comparison of stretch sensors to IMU and EMG sensors.

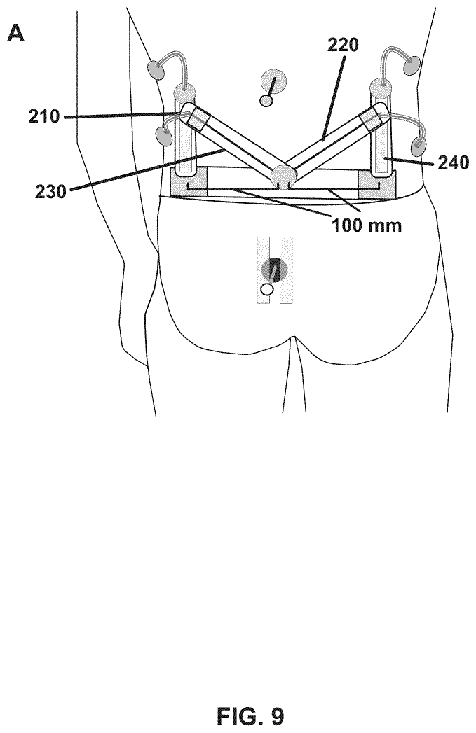

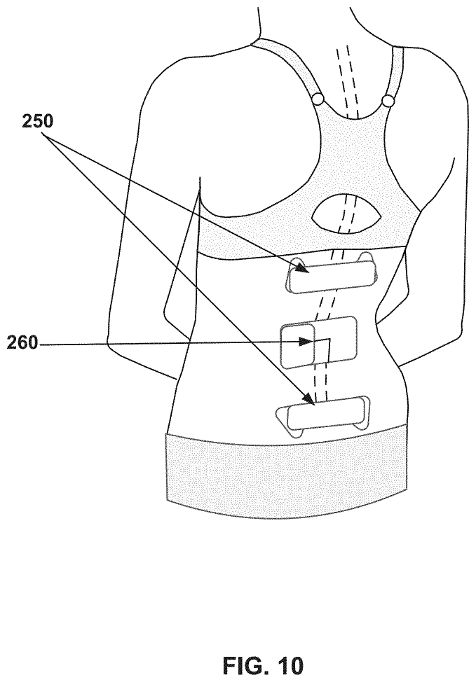

FIG. 8 shows an exemplary embodiment of an IMU sensor. In embodiments, a t-shirt 180 contains sensors 200 and a controller 190 that communicate with a smartphone 170. FIG. 9 shows an embodiment of a stretch sensor array for lumbar spine motion containing left vertical 210 and right vertical 240 portions, as well as portions that cover the left oblique 230 and the right oblique 220. FIG. 10 shows an embodiment of a combined IMU and EMG method with sensor 260 and two motion sensors 250 used to provide position feedback and monitoring muscle activity.

FIG. 11 depicts an exemplary embodiment of a two-loop sensor system as shown in elements 270 and 280. In embodiments, motion sensors provide actuatable data, such as motion biofeedback, for posture and position feedback, and pain management. In embodiments, augmented reality and physio games can be incorporated into to motion biofeedback systems.

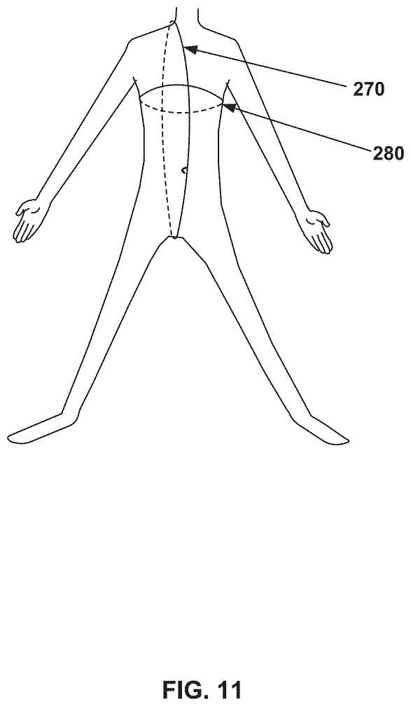

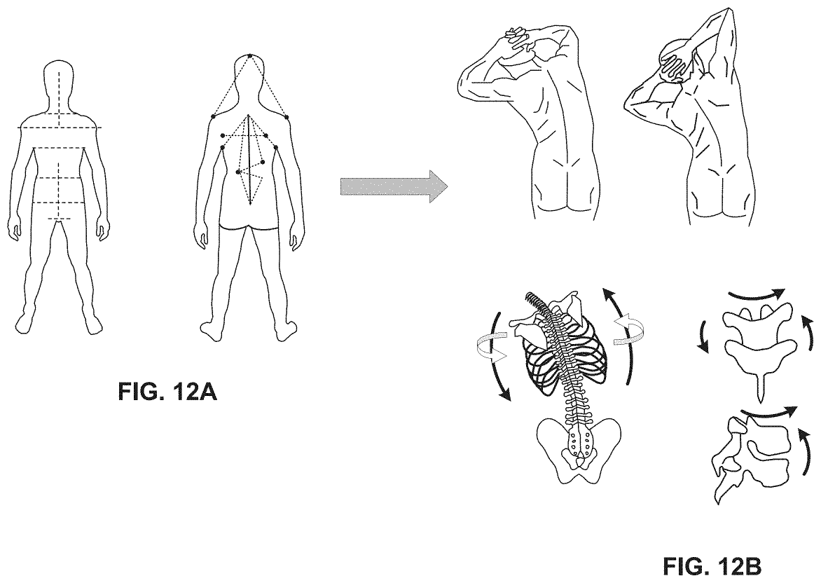

FIGS. 12A and 12B illustrate the principle of biomechanical feedback, i.e., how to link sensor data from the dorsal surface to biomechanical modeling parameters. In embodiments, to optimize biomechanical feedback requires closing the digital-physical gap between sensors and biomechanical models. FIG. 12A shows an exemplary embodiment of fringe topography of a normal subject and image analysis used to non-invasively capture spinal curve parameters. FIG. 12B shows an exemplary embodiment modeling biomechanics from the surface topography.

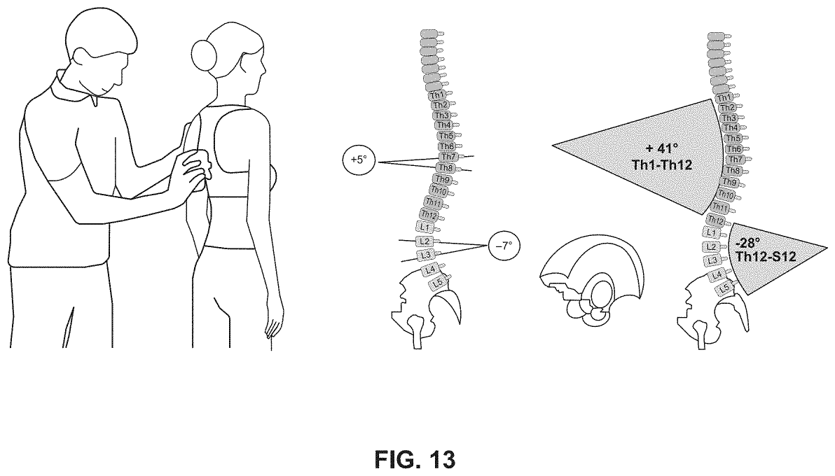

FIG. 13 shows another exemplary embodiment of non-invasive capture of spinal parameters by optical scanning. In embodiments, such method allows capturing segmental angles (left spine diagram) and regional angles (right spine diagram) of the spine. In embodiments, the captured parameters can be imported into a finite element, anatomical model of the spine to create a personalized 3D model of the spine for dynamic visual biomechanical feedback. In some embodiments, augmented reality or virtual reality can be used in the context of bio-signal monitoring, such as an EMG or an EKG. In embodiments, this can provide support for personalized and precision medicine.

To the inventor's knowledge, there are no sensor systems currently available that can be built into regular garments and processed with low complexity algorithms for analysis and visualization, bio-suits with EMG or other sensors provide monitoring data expected to be consumed with minimal processing and no biomechanical modeling. In contrast, the systems disclosed herein support spine modeling and can be integrated with other exercise methods and sensors such as weight bearing exercises, balance exercises, proprioceptive exercises, and effort-related biosignals. Specialization according to the needs of different age groups may also be implemented.

In various embodiments, in one example, a method to compute the transfer functions between sensor data, geometric distortions of the sensor array, and angular positions of the spine is provided, the method comprising applying the solutions for [A], as defined herein, [M], as defined herein, and/or a direct estimation of [A]×[M], as defined herein.

In various embodiments, in one example, a method to provide biofeedback is provided comprising monitoring deviations from recorded angular positions from a correct exercise execution in real time and at the end of an exercise.

In various embodiments, in one example, a method to incorporate visual cues and attentional cues to the 3D visualization is provided comprising conveying corrective data and reinforcing a type of biofeedback by adding correctness information to the visualization.

In various embodiments, in one example, a linear model of the dependency between four stretch sensor signals and spine angular positions is provided, comprising one or more of the following equations: Dx=(ΣSi)/2, i=1-4; Dy=(51+S3)−(S2+S4); Dz=(51+S4)−(S2+S3); X_Axis=0.5*[1+(Dx/Ax_max)*(Ax_r/Dx_r)]; Y_Axis=0.5*[1+(Dy/Ay_max)*(Ay_r/Dy_r)]; and Z_Axis=0.5*[1+(Dz/Az_max)*(Az_r/Dz_r)].

In various embodiments, in one example, a model based on the 2D projective transformation parameters and angular positions of the spine is provided, comprising one or more of the following equations:

$$H = [ \begin{matrix}{a}_{11} & {a}_{12} & {t}_{z} \\ {a}_{21} & {a}_{22} & {t}_{y} \\ {v}_{1} & {v}_{2} & v\end{matrix} ] = ( \begin{matrix}A & t \\ {v}^{T} & v\end{matrix} ) ; H = [ \begin{matrix}sR & t \\ {0}^{T} & 1\end{matrix} ] [ \begin{matrix}k & 0 \\ {0}^{T} & 1\end{matrix} ] [ \begin{matrix}λ & 0 \\ {0}^{T} & 1\end{matrix} ] [ \begin{matrix}1 & 0 \\ {V}^{T} & v\end{matrix} ] ; and [ \begin{matrix}{x}^{′} \\ {y}^{′} \\ 1\end{matrix} ] = [ \begin{matrix}{a}_{11} & {a}_{12} & {t}_{z} \\ {a}_{21} & {a}_{22} & {t}_{y} \\ {v}_{1} & {v}_{2} & v\end{matrix} ] \times [ \begin{matrix}x \\ y \\ 1\end{matrix} ] .$$

While the principles of this disclosure have been shown in various embodiments, many modifications of structure, arrangements, proportions, the elements, materials and components, used in practice, which are particularly adapted for a specific environment and operating requirements may be used without departing from the principles and scope of this disclosure. These and other changes or modifications are intended to be included within the scope of the present disclosure.

The present disclosure has been described with reference to various embodiments. However, one of ordinary skill in the art appreciates that various modifications and changes can be made without departing from the scope of the present disclosure. Accordingly, the specification is to be regarded in an illustrative rather than a restrictive sense, and all such modifications are intended to be included within the scope of the present disclosure. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, required, or essential feature or element.

As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. Also, as used herein, the terms “coupled,” “coupling,” or any other variation thereof, are intended to cover a physical connection, an electrical connection, a magnetic connection, an optical connection, a communicative connection, a functional connection, and/or any other connection. When language similar to “at least one of A, B, or C” or “at least one of A, B, and C” is used in the specification or claims, the phrase is intended to mean any of the following: (1) at least one of A; (2) at least one of B; (3) at least one of C; (4) at least one of A and at least one of B; (5) at least one of B and at least one of C; (6) at least one of A and at least one of C; or (7) at least one of A, at least one of B, and at least one of C.

What is claimed is:

1. A system to monitor and visualize spine motion comprising:
a scanning device configured to capture parameters of spinal curvature, range of motion of the spine, and regional and segmental angles of the spine;
at least one sensor configured to process signals resulting from spinal movement; and
a device configured to produce a 3D model of the spine based on the parameters captured by the scanning device and the signals processed by the at least one sensor.

2. The system of claim 1, wherein the 3D model of the spine comprises a 3D angular position of the spine.

3. The system of claim 2, further comprising an analytics device configured to interpret the signals processed by the at least one sensor.

4. The system of claim 3, further comprising a monitoring device that is configured to produce data based on the interpretation by the analytics device of the signals produced by the at least one sensor.

5. The system of claim 1, wherein the at least one sensor comprises a sensor array.

6. The system of claim 1, wherein the at least one sensor comprises at least four (4) capacitive stretch sensors.

7. The system of claim 6, wherein the at least four (4) capacitive stretch sensors are in an X-shaped configuration.

8. The system of claim 1, further comprising a display configured to produce visual feedback of the spinal movement and the 3D model.

9. The system of claim 8, wherein the display is part of a VR headset.

10. A device attached to the back of a subject to measure spine motion and spine curvature change, the device comprising:
a first capacitive stretch sensor located on the top left of the back of the subject;
a second capacitive stretch sensor located on the top right of the back of the subject;
a third capacitive stretch sensor located on the lower right of the back of the subject; and
a fourth capacitive stretch sensor located on the lower left of the back of the subject.

11. The device of claim 10, wherein the first capacitive stretch sensor, the second capacitive stretch sensor, the third capacitive stretch sensor and the fourth capacitive stretch sensor are integrated into a wearable garment or clothing.

12. The device of claim 10, wherein the first capacitive stretch sensor, the second capacitive stretch sensor, the third capacitive stretch sensor and the fourth capacitive stretch sensor are in a X-shaped configuration.

13. The device of claim 10, wherein the first capacitive stretch sensor is attached to a left shoulder area of a wearer, the second capacitive stretch sensor is attached to a right shoulder area of the wearer, the third capacitive stretch sensor is attached to a right anterior superior iliac spine area of the wearer, and the fourth capacitive stretch sensor is attached to a left anterior superior iliac spine area of the wearer.

14. A method of computing spine angle positions from a neutral posture, comprising:
measuring spinal axial reference angles in 3D (Ax, Ay, and Az); and
measuring geometric distortions of the spinal axial reference angles in 3D (Dx, Dy, and Dz).

15. The method of claim 14, further comprising determining a proportionality constant that links the spinal axial reference angles and geometric distortions of the spinal axial reference angles.

16. The method of claim 15, wherein determining the proportionality constant comprises estimating spinal axial reference angles of Ax, Ay, and Az, and estimating corresponding distortions of Dx, Dy, and Dz.

17. The method of claim 14, wherein axial angles for each 3D axis are computed by the following equations:
X_Axis=0.5*[1+(Dx/Ax_max)*(Ax_r/Dx_r)];
Y_Axis=0.5*[1+(Dy/Ay_max)*(Ay_r/Dy_r)]; and
Z_Axis=0.5*[1+(Dz/Az_max)*(Az_r/Dz_r)].

18. The method of claim 17, wherein the maximum angles are in the range of −60° to 60°.

19. The method of claim 17, wherein the equations in equations in claim 17 normalize angular values to a range between 0.0 and 1.0.

20. The method of claim 19, wherein a neutral posture is around 0.5.
